(19) United States
(12) Patent Application Publication
(10) Pub. No.: US 2010/0062461 A1
(43) Pub. Date: Mar. 11, 2010

(54) MULTIPLEX DETECTION OF CELL SURFACE RECEPTORS OR IMMOBILIZED ANTIGENS

(75) Inventor: Hui CEN, Palo Alto, CA (US)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(73) Assignee: LEAP BIOSCIENCES CORPORATION, Mountain View, CA (US)

(21) Appl. No.: 12/618,071

(22) Filed: Nov. 13, 2009

Related U.S. Application Data
(63) Continuation-in-part of application No. 12/324,554, filed on Nov. 26, 2008.
Application No. 12/324,554 is a continuation-in-part of application No. PCT/US2007/072947, filed on Jul. 6, 2007.
(60) Provisional application No. 60/819,990, filed on Jul. 11, 2006.

(51) Int. Cl. G01N 33/53

(52) U.S. Cl. 435/7.2

(57) ABSTRACT

The present invention provides an antibody-based multiplex detection method for identifying one or more epitopes in one or more samples, the method comprising the steps of: (a) contacting each of the samples comprising the epitopes with one or more antibodies specific to the epitopes to be detected; (b) washing unbound antibodies away and eluting bound antibodies from the epitope carriers to provide separate antibody eluates; and (c) detecting and quantifying antibodies in the antibody eluates to generate separate profiles of antibodies for each of the samples. The epitopes to be detected can be located intracellularly, or present on the surface of cells or an artificial surface. In general, the epitopes are derived from protein, polypeptides with or without post-translational modification, carbohydrate, nucleic acid, or lipid.

### CROSS-REFERENCE TO RELATED APPLICATIONS

This application is a Continuation-in-part application of U.S. Ser. No. 12/324,554, filed Nov. 26, 2008, which is a Continuation-in-part application of International Application No. PCT/US2007/072947, filed Jul. 6, 2007, which claims priority of U.S. Ser. No. 60/819,990 filed Jul. 11, 2006. The content of the preceding applications are hereby incorporated in their entirety by reference into this application.

### FIELD OF THE INVENTION

The present invention generally relates to proteomics. More specifically, it relates to methods for simultaneously detecting a large number of immobilized antigens.

### BACKGROUND OF THE INVENTION

Current protein detection technologies such as ELISA, Western Blotting and Antibody Array are best applied in detecting soluble proteins such as growth factors, cytokines and soluble intracellular proteins such as Src, NFκb and Iκb. Cell surface receptors can only be analyzed by flow cytometry or immunofluorescence staining. Immobilized proteins on tissue slides are normally analyzed by immunohistochemistry methods.

Flow cytometry analysis requires cells to be in suspension for conducting the analysis. Therefore, adherent cells need to be detached from the culture plates by non-enzymatic cell dissociation buffer and separated into individual cells before subjecting them to flow cytometry. However, the cell detachment step may cause change to the receptor density on the cell surface. Although immunofluorescence methods can be used to detect cell surface receptors directly without cell lifting, it is not quantitative. So it will be desirable to develop a new quantitative method which can measure cell surface receptor density of adherent cells without detachment.

In addition, the multiplicity of both immunofluorescence and flow cytometry methods are determined by the availability of non-interfering fluorophores. Since there are no more than 4 of such fluorophores currently available, multiplex antigen detection is limited to 4 in both methods. Both immunofluorescence and flow cytometry are also popularly used to detect intracellular proteins after immobilizing the proteins via cell fixation. Immunohistochemistry methods are used for detecting desired antigens on tissue slides. Immunohistochemistry methods are also non-quantitative and are single-plex. Since most biological networks usually involve 10 to 100 protein members, it is highly desirable to develop a method that can simultaneously detect a large number of proteins for their expression and modification without disturbing their biological state.

### SUMMARY OF THE INVENTION

This invention provides a method for simultaneously detecting a large number of cell surface or immobilized antigens. In the scenario that the immobilized antigens are one or multiple cell surface receptors, this invention provides a method for detecting receptor density on cell surface without detaching adherent cells from the culture surface. In the scenario that the immobilized antigens are cellular proteins, this invention provides a method for simultaneously detecting one or multiple cellular proteins for their expression and/or modification. In the scenario that the immobilized antigens are derived from soluble antigens in a biological sample, this invention provides a method for simultaneously detecting one or multiple soluble antigens for their expression and/or modification. In addition, this invention is capable of detecting multiple epitopes of the same molecules simultaneously or epitopes on different proteins even when they are in complex of each other. This capability is lacking in current Antibody Array-based multiplex detection methods.

Current Antibody Array is essentially a particle-capture based detection technology, wherein each antibody immobilized in a specific position on a planar array or a specific bead in the suspension array is used to capture its corresponding antigen as a particle in a sample and derive the amount of antigen by quantifying the amount of the captured particle. This format can only apply to the scenario that all antigens to be detected are present as separate particles. It is not applicable when two antigens to be detected are in complex of each other, therefore becoming a single particle. This format also is limited to detecting only a single epitope per antigen due to the nature of being a single particle per antigen.

The present invention is an epitope-stamping based detection technology. Antibodies are supplied in soluble form to bind corresponding epitopes on cell surface or immobilized antigens. After removing unbound antibodies, the bound antibodies which can be considered to be “epitope-stamped” are eluted from the antigens and quantified by epitope array, immunoassay or mass spectroscopy-based methods well known in the art. Various antibody elution methods are known in the art, for example, there are methods of eluding antibody protein adherent to the red cell surface in order to determine their serological specificity [1, 2, 3]. Antibody stripping which is equivalent to antibody elution has also been used in Western blotting and immunohistochemistry for sequential detecting and staining of multiple antigens [4, 5]. Although eluting antibodies from antigens has been practiced before, it has never been used as a detection method for quantifying expression and modification of one or more target epitopes or antigens as disclosed herein. In summary, the present invention can detect any cell surface or immobilized antigen even when they are in a complex. The present invention can also detect multiple epitopes on the same antigens.

### BRIEF DESCRIPTION OF THE DRAWINGS

A detailed description of various aspects, features and embodiments of the present invention is provided herein with reference to the accompanying drawings, which are briefly described below. The drawings are illustrative and are not necessarily drawn to scale. The drawings illustrate various aspects, or features, of the present invention and may illustrate one or more embodiment(s) or example(s) of the present invention in whole or in part. A reference numeral, letter, and/or symbol that are used in one drawing to refer to a particular element or feature may be used in another drawing to refer to a like element or feature.

FIG. 1 shows multiple types of antibodies in the eluate can also be simultaneously fractionated and detected by an epitope array, a form of protein array (LEAP Multiplex Detection Techonology).

FIG. 2 shows detection of mouse anti-EGFR antibody eluted from cells surface of Hela and A431 cells.

FIG. 3 shows simultaneous detection of EGFR and ErbB2 on cell surface of A431 and Hela cells and sequential detection of anti-EGFR and anti-ErbB2 antibodies in the eluate.

### DETAILED DESCRIPTION OF THE INVENTION

In relation to the brief summary and the description, it will be understood that a word appearing in the singular encompasses its plural counterpart, and a word appearing in the plural encompasses its singular counterpart, unless implicitly or explicitly understood or stated otherwise. Further, it will be understood that for any given component described herein, any of the possible candidates or alternatives listed for that component, may generally be used individually or in any combination with one another, unless implicitly or explicitly understood or stated otherwise. Additionally, it will be understood that any list of such candidates or alternatives, is merely illustrative, not limiting, unless implicitly or explicitly understood or stated otherwise. Still further, it will be understood that any figure or number or amount presented herein in connection with the invention is approximate, and that any numerical range includes the minimum number and the maximum number defining the range, unless implicitly or explicitly understood or stated otherwise. Additionally, it will be understood that any permissive, open, or open-ended language encompasses any relatively permissive to restrictive language, open to closed language, or open-ended to closed-ended language, respectively, unless implicitly or explicitly understood or stated otherwise. Merely by way of example, the word “comprising” may encompass “comprising”, “consisting essentially of”, and/or “consisting of” type language.

Various terms are generally described or used herein to facilitate understanding of the invention. It will be understood that a corresponding general description or use of these various terms applies to corresponding linguistic or grammatical variations or forms of these various terms. It will also be understood that a general description or use of a corresponding general description or use of any term herein may not apply or may not fully apply when the term is used in a non-general or more specific manner. It will also be understood that the invention is not limited to the terminology used herein, or the descriptions thereof, for the description of particular embodiments. It will further be understood that the invention is not limited to embodiments of the invention as described herein or applications of the invention as described herein, as such may vary.

### Epitopes and Epitope Carriers

Generally, the term “epitope” refers to a site on a large molecule against which an antibody was produced and to which it binds. An epitope can be located on peptide, protein, carbohydrate, lipid, nucleic acid or hybrid of these molecules. The epitope is a part of an antigen. An antigen can have multiple epitopes. The epitope and antigen can be from any species. The antigen can be a cell surface receptor, cell surface protein, extracellular protein, intracellular protein, carbohydrate, nucleic acid, lipid, small organic molecules, or other molecules. The epitope may also comprise post-translational modification of a peptide or modification of carbohydrate, nucleic acid, lipid, or small organic molecules. Examples of post-translational modification include, but are not limited to, phosphorylation, acetylation, alkylation, amidation, biotinylation, formylation, gamma-carboxylation, glutamylation, glycosylation, glycation, glycylation, hydroxylation, iodination, isoprenylation, lipoylation (e.g. prenylation, myristoylation, farnesylation and geranylgeranylation), ADP-ribosylation, heme attachment, flavin attachment, oxidation, palmitoylation, pegylation, phosphatidylinositol attachment, phosphopantetheinylation, polysialylation, tRNA-mediation addition of amino acids such as arginylation, pyroglutamate formation, sulfation, selenoylation, the covalent linkage to the ISG15 protein, sumoylation, ubiquitination, neddylation, citrullination, deamidation, and eliminylation.

Generally, the term “epitope carrier” refers to a substance carrying or expressing a plurality of epitopes that are capable of interacting with antibody molecules in a solution. In one embodiment, the epitope carrier may be a biological surface immobilized with antigens. The epitope carrier or carriers may be substances or objects wherein at least part of the surface of the substances or objects is a biological surface immobilized with antigens. Generally, the term “biological surface” refers to a surface or matrix on which a plurality of antigens each bearing one or more epitopes are or can be immobilized either non-covalently or covalently. Examples of biological surfaces include, but are not limited to, the exterior or interior surface of a cytoplasmic membrane, cell organelle membrane, a tissue, a tissue section, live cells, apoptotic cells, dead cells or fixed cells as long as the epitope to be detected is intact or can be restored. Cells can be fixed by a number of agents and methods currently known in the art (e.g. formaldehyde fixation). The cell may be prokaryotic or eukaryotic. The cell can be an animal cell, a plant cell, a bacteria cell, a yeast cell or a fungus cell, merely by way of example. When the cell is of animal origin, it may be a cell from any vertebrate or any invertebrate animal. Examples of vertebrate animals include, but are not limited to, humans, mice, rats, pigs, cows, monkeys, rabbits, chickens, and the likes. Examples of invertebrate animals include, but are not limited to, drosophila, zebra fish, worms and the likes. The cell may be an adherent cell such as HeLa, PC3, Cos cell or the like, or maybe a suspension cell such as Jurkat, HL-60 cell, and/or the like, merely by way of example. The cell may belong to a primary cell type or to an immortalized cell type.

The “epitope carrier” may be whole cell or part of cells (such as cell sections) as present on tissue sections on tissue slides. Tissue slides with either a frozen tissue section or formalin-fixed paraffin-embedded tissue section can be directly used for detecting various cellular proteins including intracellular and extracellular proteins by detecting epitopes on these cellular proteins since cellular proteins have been immobilized on the slide and exposed due to tissue sectioning. Additional fixation steps may be implemented if the cellular proteins to be detected are not fully immobilized.

In another embodiment, the “epitope carrier” can be artificial biological surface such as surface of a non-biological material wherein the material may be in the physical form of a well, a plate, a particle, a slide, a bead, a fiber, a matrix, a porous structure, a stick, a membrane, a chip, or the like, and the material may be selected from the list of sepharose, agarose, latex, dextran, lipid monolayer, lipid bilayer, metal, metal oxide, glass, ceramic, quartz, plastic, silicon, polyacrylamide, polystyrene, polyethylene, polypropylene, polymer, a colloid, polycarbonate, polytetrafluoroethylene, silicon oxide, silicon nitride, cellulose acetate membrane, nitrocellulose membrane, nylon membrane and polypropylene membrane, amorphous silicon carbide, castable oxides, polyimides, polymethylmethacrylates, and silicone elastomers and/or the like.

To prepare the epitope carrier using an artificial biological surface, antigens are immobilized onto an artificial surface. The immobilization process should generally not be too harsh to change the conformation of the antigens. The antigen immobilization should be strong enough so that the antigens remain immobilized during the wash step and preferably the elution step. The association between the antigens and the substance or object material of the surface may be due to non-covalent interaction, covalent bonding, or a combination thereof. Various conventional methods used for antigen immobilization can be applied. Methods of protein immobilization that preserve protein functionalities are well known. Examples of such methods include covalent attachment of proteins and immobilization of biotinylated protein onto streptavidin-coated surfaces (Ruiz-Taylor et al., PNAS 2001, 98:852-857); covalent attachment of proteins to a surface functionalized with amine-reactive groups (MacBeath et al., Science 2000, 289:1760-1763; Zhu et al., Nat Genet. 2000, 26:283-289; Arenkov et al., Anal Biochem 2000, 278:123-131); and covalent immobilization of oxidized glycoproteins onto surface functionalized with aldehyde-reactive groups. Additional examples of covalently or non-covalently immobilizing proteins onto a surface can be found in the following references: Kenausis et al., J Phys Chem B 2000, 104:3298-3309; MacBeath, G. et al., J. Am. Chem. Soc. 1999, 121:7967-7968; Hergenrother et al., J. Am. Chem. Soc. 2000, 122:7849-7850; Falsey et al., Bioconjugate Chem. 2001, 12:346-353; Houseman et al., Nat. Biotechnol. 2002, 20:270-274; Wang et al., Nat. Biotechnol. 2002, 20:275-280; and Sun et al., Bioconjugate Chem. 2006, 17:52-57; Prime et al., Science 1991, 252:1164-1167.

In another embodiment, to detect epitopes present on intracellular proteins in culture cells, the immobilization of intracellular proteins can be achieved by fixation of cells. Various cell fixation agents such as formalin, glutaraldehyde, methanol and cell permeablization agents described elsewhere including in Morphology Methods: Cell and Molecular Biology Techniques by Ricardo V. Lloyd Humana Press, 2001 and Cytometric Analysis of Cell Phenotype and Function by Desmond A McCarthy (ed.) and Marion G. Macy (ed.) Cambridge University Press, 2001 can be used to fix and permeabilize cells to achieve immobilization and exposure of epitope for detection by antibody using this invention. For adherent culture cells, cells can be fixed onto the culture surface directly followed by permeabilization. For suspension cells, cells can be fixed and permeabilized using the same method for detecting intracellular protein by flow cytometry.

The epitope carrier or carriers should be readily separated from a solution containing antibody molecules by conventional separation methods such as centrifugation, filtration, precipitation, magnetic field, affinity capture, or aspiration of the liquid.

### Antibody Binding

In one embodiment, antibodies or their antigen-binding sites are supplied in excess over the amount of epitope to be detected to ensure the antibody amount quantified in the eluate is proportional to the epitope amount present in the sample. Antibodies or their antigen-binding sites can be constructed or formulated into various forms well-known in art. Hence, the antibodies can be from any species and in any form such as natural or artificially engineered, partial or complete or fusion (chimera), or in phage display form as long as it contains the antigen-binding site.

The antibodies used in the present invention can be monoclonal antibodies or polyclonal antibodies. For detecting multiple epitopes on a single antigen, a monoclonal antibody is preferred since it may minimize the chance of steric hindrance created by the antibodies against different epitopes on the same antigen.

Once a suitable antibody solution is prepared, it is incubated in an appropriate vessel with the epitope carrier of each sample for a time sufficient for the antibody to bind to its epitope in the sample. In one embodiment, the incubation time is from about 10 minutes to about 2 hours. The incubation temperature is preferably from around 4° C. to around 37° C. Optionally, to minimize non-specific binding of antibody to the biological surface, a blocking solution containing a suitable amount such as 1-10 mg/mL of carrier protein such as BSA, IgG, milk proteins or other proteins is incubated with the epitope carriers for about 10 minutes to about 2 hours at a temperature from about 4° C. to about 37° C. before incubating with the antibody solution. Preferably, the antibody solution is made with high concentration of carrier proteins. The carrier proteins are preferably to contain IgG or other molecules capable of blocking Fc receptors present on the epitope carrier in the sample.

To detect receptors present on cell surface, live cells or fixed cells without permeabilization can be used since the intact cell membrane serves as barrier preventing the antibody from entering cells which would result in detecting receptors present inside cells as well. When using live cells, incubation of antibody with cells needs to be performed at cold temperatures to prevent receptor internalization. If inhibitors of endocytosis of receptors are used, the incubation of antibody with cells may be conducted at room temperature. However, for fixed and non-permeabilized cells, the incubation can be conducted at room temperature.

Growth factor receptors play pivotal role in cell proliferation, evasion of apoptosis, angiogenesis, migration and metastasis. For example, over-expression of growth factor receptor EGFR and ErbB2 have been found in many human cancers including bladder, breast, lung, and colon cancers. Over-expression of HGF receptor c-Met was found in 67% of adenocarcinomas, 60% of carcinoids, 57% of large cell carcinomas, 57% of squamous cell carcinomas. Over expression of PDGF receptors are associated with tumor progression in breast cancer and glioblastomas. VEGF receptor and its family members are key protein regulating cancer angiogenesis. These growth factor receptors currently serve as important cancer drug targets for successful cancer drugs such as Herceptin. However, Herceptin is only effective on ErbB-2 positive breast cancers, applicable to only ⅓ of breast cancer patients. With the increasing number of therapeutic agents targeted at growth factor receptors available, a multiplex detection method for studying expression of these receptors simultaneously on cancer tissue would provide a new methodology for efficient identification of abnormal expression of a variety of cell surface molecules related to cancer or other diseases. Hence, the present invention can be used by doctors to prescribe specific antibody therapy against one or more particular receptors present on a particular patient's tumor cells for effective personalized therapy.

Various methods used in immunohistochemistry to expose epitopes can be used to increase detection of epitopes by antibody molecules. Multiple antibodies detecting multiple antigens or biomarkers used in current immunohistochemistry staining can be combined to detect these multiple biomarkers using a single tissue slide using the method of this invention. Generally, the term “biomarkers” refers to a characteristic, or a combination of characteristics, that can be objectively measured and evaluated as an indicator of normal biological processes, pathogenic processes, or a pharmacological response to a therapeutic intervention. The simultaneous detection of multiple biomarkers in a single sample by the present invention eliminates sample variation introduced by multiple samples for multiple biomarkers in current immunohistochemistry format of one biomarker per assay. Examples of useful biomarkers include, but are not limited to, ErbB-2, EGFR, c-Kit, ER, PR, PSA, ACTH (Adrenocorticotrophin), Actin, ALK-1, Alpha-1 Fetoprotein (AFP), Alpha-1-Antichymotrypsin (ACT), Alpha-1-Antitrypsin (AAT), BCA-225 (Breast Carcinoma Antigen), bcl-2, bcl-6, Beta-ratenin, CA 19-9, CA-125, Calcitonin, Carcinoembryonic Antigen (CEA), CD10 (CALLA), CD138 (Syndecan-1), CD15, CD1a, CD20, CD21, CD23, CD3, CD30, CD34, CD43, CD45 (LCA), CD45R, CD450, CD5, CD56, CD57, CD61, CD68, CD79, CD8, CD99, Chromogranin, Collagen Type-IV, COX-2 (cyclooxygenase 2), Cyclin D1, Cytokeratin (34betaE12), Cytokeratin (AE1), Cytokeratin (AE3), Cytokeratin (Pan), Cytokeratin 14, Cytokeratin 19, Cytokeratin 20, Cytokeratin 5 & 6, Cytokeratin 7, Cytokeratin 8, Desmin, E-Cadherin, EMA (Epithelial Membrane Antigen), Factor VIII Related Antigen, Fascin, FSH (Follicle Stimulating Hormone), Galectin-3, Gastrin, OH (Growth Hormone), Glial Fibrillary Acidic Protein (GFAP), Glucagon, Glycophorin A, Granzyme B, hCG (human Chorionic Gonadotrophin), Inhibin, Insulin, Kappa light chain, Keratin, Ki-67 (30-9), Lambda light chain, LH (Luteinizing Hormone), MART-1/melan A, WEI, MSH, Myeloperoxidase, Myosin (Smooth Muscle-specific), Neurofilament, NSE (Neuron Specific Enolase), p27Kip1, p53, Pax-5 (BSAP), PLAP (Placental Alkaline Phosphatase), Prolactin, PSAP (Prostatic Acid Phosphatase), S100, Somatostatin, Spectrin, Synaptophysin, TAG-72, TdT (Terminal Deoxynucleotidyl Transferase), Thyroglobulin, Thyroid Transcription Factor-1 (TTF-1), TRAcP (Tartrate-resistant Acid Phosphatase), Tryptase, TSH (Thyroid Stimulating Hormone), Villin, Vimentin, Von Willebrand Factor, ZAP-70. Biomarkers can also be an infectious disease agent such as virus and bacteria, for example, Helicobacter pylori, HHV-8 (Human Herpes Virus Type 8), etc. Biomarkers can also be a marker of a specific organelle or cell, for example, Lysozyme, Macrophage, Renal Cell Carcinoma (RCC), Melanosome, etc.

Examples of additional biomarkers or antigens and their antibodies which can be used in the present invention can be found on the websites of following companies: Abcam, AbD Serotec, Abgent, Advanced Targeting Systems, Antigenix America Inc, Assay Designs/Stressgen Bioreagents, Atlas Antibodies, Ayes Labs, Inc., Bachem, BD Biosciences, Beckman Coulter, Bender MedSystems, Bethyl Laboratories, Biocare Medical, BioCytex, BioGenex, BioLegend, BioVendor Laboratory Medicine, Inc., BioVision, Cayman Chemical, Cell Sciences, Cell Signaling Technology, Covance Research Products Inc, Dako, Epitomics, Inc. Fitzgerald Industries International, GeneTex, GenWay Biotech, Inc., HyTest Ltd., Imgenex, Immundiagnostik, Invitrogen, Leinco, Lab Vision, Meridian Life Science, Inc., Millipore (CHEMICON/Upstate/Linco), Miltenyi Biotec, Molecular Probes (Invitrogen), Novus Biologicals, Pro Sci Incorporated, QED Bioscience Inc., R&D Systems, Rockland Immunochemicals, Inc., Santa Cruz Biothechnology, Sigma-Aldrich, Spring Bioscience, Stem Cell Technologies, Inc., Thermo Scientific Pierce Antibodies, Ventana Medical Systems, and Vector Labs.

Once the antibody is bound to its epitope, the remaining antibody solution is separated from the epitope carrier using any of the known procedures used for separating a liquid from a solid or a semi-solid. Examples of such methods include centrifugation of the solid-liquid mixture and aspiration of the liquid phase using a vacuum device. For example, the culture medium may be removed from adherent cells by aspiration. For cells in a suspension, the culture medium may be removed by centrifugation. Optionally and preferably, the separated antibody-bound epitope carrier is further washed one or more times with PBS buffer or another solution that does not disrupt antibody/epitope binding to remove any residual unbound antibody from the epitope carrier. Alternatively, the antibody-bound epitope carrier can be separated from the liquid by filtration. The filtration can be achieved through applying vacuum to remove liquid from the epitope carrier. Filtration can also achieved through centrifugation with spin columns to remove liquid from the epitope carrier.

### Antibody Elution

In general, the term “eluate” refers to the solution recovered after the elution step.

Antibody molecules bound to the epitope carrier are dissociated from the epitope by incubating the antibody-bound epitope carrier in an antibody elution solution at appropriate temperature such as from about 4° C. to about 37° C. for a sufficient amount of time such as from about 5 minutes to about 30 minutes. The antibody and epitope are usually bound by physical interactions such as hydrophobic interaction (Van der Waals interaction), hydrogen bonding, electrostatic interaction, or a combination thereof. These forces are typically strongest when the antibody-epitope complex is in an aqueous buffer with physiological pH and ionic strength. Thus, any deviation in pH or ionic strength or both pH and ionic strength from their physiological states will weaken the antibody-epitope interaction. In addition, certain agents such as chaotropic agents are commonly used to weaken physical interactions between the antibody and the epitope. One of ordinary skill in the art could readily choose a suitable elution solution to use depending on the nature of the interaction between the antibody and the epitope.

In general, a suitable elution solution for the present invention is one that is capable of weakening the antibody and epitope interaction without chemically damaging the structure of the antibody. A suitable elution solution should also preferably not extract the epitopes off the epitope carriers. Typically, a suitable elution solution may be a buffer having a pH substantially different from the physiological pH such as a pH of 2-3 or a pH of 9.5-11.5. For example, a suitable elution solution is a pH 2-3 or pH 9.5-11 buffer comprising a chaotropic agent. When the epitope carrier is a cell, an additional salt such as NaCl at around 150 mM is also a component of the elution solution to maintain the cell in an isotonic state. One of ordinary skill in the art would readily derive a suitable elution solution. Various elution solutions used to elute antibody from antigen on affinity column can be used. An example of elution solution for cell-based epitope carriers is a pH 2.0-3.0 buffer comprising 50-100 mM glycine and 150-500 mM NaCl. This buffer effectively dissociates most protein-protein binding interactions without permanently affecting protein structure. Table 1 lists examples of antibody elution solutions. Some of them are suitable for eluting antibodies from live cells as the epitope carrier.

TABLE 1
List of Elution Solutions for Antibody Dissociation

| | Elution Solution |
|---|---|
| | (150 mM NaCl is added for elution solution |
| | for cell-based receptor |
| Elution Condition | carriers to maintain isotonic condition of the cells) |
| Low pH | 100 mM glycine HCl, pH 2.5-3.0 |
| | 100 mM acetic acid, pH 2.0-3.0 |
| | 100 mM citic acid, pH 3.0 |
| High pH | 50-100 mM triethylamine or triethanolamine, pH 11.5 |
| | 150 mM ammonium hydroxide, pH 10.5 |
| | 0.1 M glycine NaOH, pH 10.0 |
| Ionic strength | 5 M lithium chloride |
| | 3.5 M magnesium or potassium chloride |
| | 3.0 M potassium chloride |
| | 2.5 M sodium or potassium iodide |
| | 0.2-3.0 M sodium thiocyanate |
| | 0.1 M Tris-acetate with 2.0 M NaCl, pH 7.7 |
| Chaotropic effect | 2-6 M guanidine HCl |
| | 2-8 M urea |
| | 1.0 M ammonium thiocyanate |
| | 1% sodium deoxycholate |
| | 1% SDS |
| | 10% dioxane |
| | 50% ethylene glycol, pH 8-11.5 |
| Low pH & Ionic | 50 mM glycine HCl, pH 2.5-3.0, 0.5M NaCl |
| strength | |

Following antibody dissociation from the epitopes, the eluate is separated from epitope carriers using a suitable means such as centrifugation, pipetting, aspiration or the like. If the elution solution used is either acidic or alkaline, the eluate may need to be immediately brought to neutrality to avoid antibody damage using either a concentrated alkaline solution or a concentrated acidic solution. For example, if a pH 2.5-3 elution solution is used in antibody dissociation, a 1 M pH 8.5 Tris or Hepes buffer may be used to neutralize the eluted antibody solution. On the other hand, if the eluate comprising a high salt concentration is used, the eluate is usually desalted via dialysis or dilution, for example, to avoid protein precipitation. The eluate may be concentrated to a smaller volume, if necessary, using any of the suitable known concentration methods such as membrane filtration, evaporation using a Speed-Vac and lyophilization, or protein precipitation, etc.

### Simultaneously Quantification of Multiple Epitopes

Since many epitopes can be present on a large molecule, the present invention can be used to simultaneously quantify multiple epitopes on a molecule. There are epitopes that are constitutively present on the molecule and other epitopes whose presence or absence is associated with the activation state of the molecule. For example, the epitope present on Akt protein in both activated and un-activated states is a constitutive epitope. Using an antibody against the constitutive epitope allows quantification of total Akt protein present in the sample. The epitope containing phosphorylated threonine at the 308 amino acid position of Akt (Phosph-Akt Thr308) is an activation epitope since it is only present on Akt at the activated state. Using antibody against the activation epitope Phosph-Akt Thr308 allows quantification of Akt in this activated state. Simultaneous detection of both constitutive epitope and activated epitope of a signaling protein such as Akt allows generating normalized signal ratio between the activated epitope and the constitutive epitope. This ratio can be used to accurately compare Akt activation level among different samples. Another example of activation epitope is the C-terminal portion of NFκB2 p100 which is degraded upon activation of NFκB2 p100.

Accordingly, the present invention can be an effective tool to monitor expression and/or activation of a variety of proteins related to one or more biological pathway, protein family or disease. For example, for the NFκB pathway, this invention allows simultaneous detection of multiple members of NFκB pathway for both their total protein expression and one or more activated forms. These members and their activation form can include p38 MAPK and p38 MAPK (phosphor-Tyr180/182), JNK and JNK (phosphor-Thr183/185), TAK1 and TAK1 (phosphor-Thr184), Akt1 and Akt1 (phosphor-Ser473), IkB and IkB (phosphor-Tyr32/36), IKKalpha and IKKalpha (phosphor-Tyr32), NFκB, NFκB (phosphor-Ser536) and NFκB (phosphor-Ser276). Additonal examples of biological pathways include, but are not limited to pathways of Chromatin Regulation/Remodeling, Apoptosis, Autophagy, Cellular Senescence, PI3K/Akt Signaling, Translational Control, Ca, cAMP & Lipid Signaling, Cell Cycle/Checkpoint, DNA Damage, Jak/Stat Signaling, TGF-β/Smad Signaling, Lymphocyte Signaling, Neuronal Signaling, Angiogenesis, Vesicle Trafficking, Cell Motility, Cytoskeletal Signaling, Cell Adhesion Signaling, Glucose Metabolism, Wnt Signaling, Hedgehod Signaling, Notch Signaling, Nuclear Receptor Signaling, Protein Folding & Stability, Cancer Drug Resistance & Metabolism, Dendritic & Antigen Presenting Cell Signaling, EGF/PDGF Signaling, Embryonic Stem Cell Signaling, Endothelial Cell Signaling, Epithelial to Mesenchymal Transition, Genome Stability, DNA Repair, DNA Replication, Hematopoiesis, Hematopoietic Stem Cell Signaling, HIV Infection and Host Response, Hypoxia Signaling, Inflammatory Response, Innate & Adaptive Immune Response, Insulin Signaling, Interferon Response, Lipoprotein Signaling, Cholesterol Metabolism, Mesenchymal Stem Cell Signaling, Cell Development & Differentiation, Mitochondria Signaling, Neurogenesis, Neuronal Stem Cell Signaling, Nitric oxide Signaling, Osteogenesis, Oxidative Stress & Antioxidant Defense, p53 Signaling, Stem Cell Signaling, Stress Response to Cellular Damage, T-cell activation, B-cell activation, T Cell Anergy & Immune Tolerance, Th1, Th2, Th3, Th17 for Autoimmunity & Inflammation, Dendritic Cell Signaling, NK Cell Signaling, B Cell Signaling, T Regulatory Cell Signaling, T Helper Cell Differentiation, Toll-like Receptor Signaling, Toxicology & Drug Resistance, Tumor Metastasis, Ubiquitynation and Unfold protein Response.

Examples of protein families include, but are not limited to, Cell Surface Markers, Cytokine Receptors, Chemokines Receptors, Growth Factor Receptors, T Cell Receptors, Common Cytokines, Chemokines & Receptors, Inflammatory Cytokines & Receptors, Drug Transporters, Cytoskeleton Regulators, Drug Metabolism Enzymes, Epigenetic Chromatin Modification Enzymes, Epigenetic Chromatin Remodeling Factors, Extracellular matrix and Adhesion Molecules, Growth Factors, Heat Shock Proteins, Homeobox Proteins, Acute Phase Proteins, Matrix Proteins, Death Receptors, Housekeeping Proteins, Interferon & Receptors, Neuroscience Ion Channels & Transporters, Neurotransmitter Receptors & Regulators, Neurotrophin and Receptors, Nuclear Receptors & Coregulators, Oncoproteins, Tumor Suppressor Proteins, Protein Kinases, Protein Phosphatases, Stem Cell Transcription Factors, Transcription Factors, Costimulatory molecules, MHC Antigens, TNF Ligand & Receptor family, Tyrosine Kinase/Adaptors. Examples of diseases include cancer, neurological disorders, cardiovascular disease, Alzheimer, Parkinson, Asthma, Atherosclerosis, Diabetes, Breast Cancer, Colon Cancer, Gastric Cancer, Liver Cancer, Lung Cancer, Obesity, Prostate cancer, Arthritis, Autoimmune Disease, etc.

In another embodiment, the present invention can be used to profile activation of multiple biological pathways by simultaneous detecting the expression and/or activation form of one or more signaling molecules from each pathway. For example, a multiplex detection kit can be constructed for monitoring activation of the following 5 signal transduction pathways: Akt, MAPK, p38, JNK and NFκB. This pathway activation profiling kit can contain antibodies detecting the following proteins and their phosphorylated epitope: Akt1, Akt1 (phosphor-Ser473), p44/42, Phospho-p44/42 MAPK (Thr202/Tyr204), p38α, Phospho-p38α MAPK (Thr180/Tyr182), MEK1, Phospho-MEK1/2 (Ser217/221), JNK, Phospho-SAPK/JNK (Thr183/Tyr185), IκB and IkB (phosphor-Tyr32/36). Other examples of pathway activation profiling kits include, but are not limited to, Cancer Pathway Activation Profiling Kit to determine the state of proliferation, angiogenesis, and metastasis of each cancer specimen; Pathway Activation Profiling Kit for GPCR Signaling or Stress & Toxicity Signaling to determine which downstream pathways are activated in a particular biological setting.

### Profiles of Antibodies in the Antibody Eluates

One of ordinary skill in the art would readily employ one or more detecting and quantifying methods to generate profiles of antibodies in the antibody eluates obtained above. The antibodies in the eluate can be detected and quantified either sequentially or simultaneously. However, simultaneous detection is preferred due to its efficiency and elimination of assay variation due to uneven sample aliquoting. In one embodiment, quantifying each epitope-specific antibody in the antibody eluates comprises detecting species-specific antibody. In another embodiment, quantifying the antibodies in the antibody eluates comprises uses of mass spectroscopy. In another embodiment, detecting antibodies in the antibody eluates comprises epitope-specific detection (e.g. epitope array). The antibody detection can use label-free detection method such as the label-free technology by ForteBio (Menlo Park, Calif.) or various fluorescence or enzyme labeling technologies well known in the art, such as those described in WO/2003/031591 by Li Shen and PCT/US2007/072947 by Hui Cen, etc.

If there is only a single type of antibody in the eluate, the antibody in the eluate can be detected by immunoassay specific to the species of the antibody. For example, if mouse anti-human EGF receptor monoclonal antibody is used to detect EGF receptor present on cell surface of human cells, the eluted mouse monoclonal antibody can be detected by ELISA specific for mouse antibody using a pair of anti-mouse antibodies. If more than one type of antibody is in the eluate and each type is different from the other type in term of the species, they can also be quantified by species-specific ELISA separately or simultaneously if multiple non-interfering readout is provided. For example, if mouse anti-EGF receptor monoclonal antibody is used to detect the EGF receptor present on cell surface of human cells and rabbit anti-ErbB2 monoclonal antibody is used to detect ErbB2 present on cell surface of the same human cells, the mouse monoclonal antibody and rabbit monoclonal antibody can be quantified by separate ELISA, one by a pair of anti-mouse antibodies and another by a pair of anti-rabbit antibodies. If the detecting antibody for each species (mouse or rabbit antibody) is labeled with different readout system such as one is conjugated with HRP and the other is conjugated with AP, both species of antibodies can be detected simultaneously when supplied with AP-specific and HRP-specific substrates (R&D Systems, Minneapolis, Minn.).

Preferably a normalization marker is included in the multiplex detection. For detecting receptors present on cell surfaces, the normalization marker can be any extracellular protein whose expression does not change upon the stimulation used in the experiment. Such extracellular proteins can be collagen, fibronectin, laminin and proteoglycan. If detecting intracellular proteins, the normalization marker can be Actin, GAPDH or other house keeping proteins or double stranded DNA or one of histone molecule since the amount of these molecules tend to be constant and proportional to the number of cells. These normalization markers can be used for normalizing to the cell number. If the normalization marker is immobilized, the antibody against the normalization marker can be used to measure the normalization marker together with antibodies against other epitopes to be detected and quantified using epitope array including the epitope of the normalization marker. If the normalization marker can be eluted from the sample during elution step, immunoassay directly quantify the normalization marker can be implemented. In addition, cell number, total protein amount, total nucleic acid amount or DNA amount can also be used as sample normalization marker.

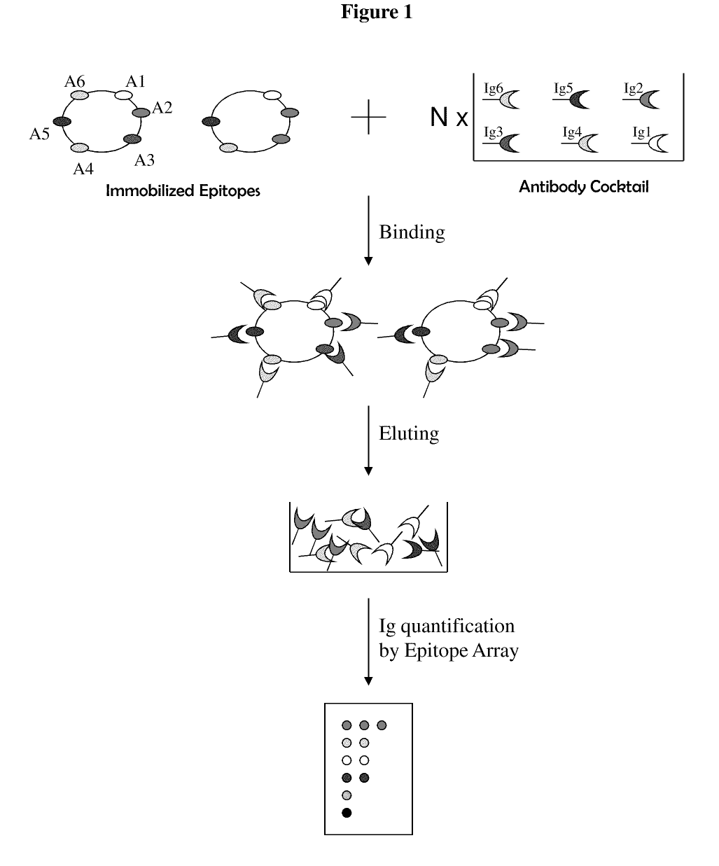

Multiple types of antibodies in the eluate can also be simultaneously fractionated and detected by an epitope array, a form of protein array (FIG. 1). The advantage of using epitope array is there is no limitation of multiplicity in the assay. An epitope array is a protein array and an antigen array. Therefore various conventional protein array or antigen array-based antibody profiling methods described elsewhere can be applied here. Examples of these methods are Protein Arrays: Methods and Protocols edited by Eric T. Fung, Humana Press, 2004 and Hueber W. et al., Antigen microarray profiling of autoantibodies in rheumatoid arthritis, Arthritis Rheum. 2005 September; 52(9):2645-55. 2005. Example of suspension protein/antigen array preparation and antibody detection method is described by Dias, D. et al., Optimization and Validation of a Multiplexed Luminex Assay To Quantify Antibodies to Neutralizing Epitopes on Human Papillomaviruses 6, 11, 16, and 18. Clinical and Diagnostic Laboratory Immunology 12(8)' 959-969, 2005.

In one embodiment, the epitope array can be planar or suspension array. For planar array, a specific epitope is immobilized on a pre-determined position of the array. For suspension array, each bead with a unique identity is coated with a specific epitope. A mixture of beads with a unique epitope on each type of bead can then be used to quantify multiple types of antibodies in the eluate. An example of such suspension array and its detection system is Luminex system from Luminex Corporation (Austin, Tex.) in which each type of bead is coded with a specific color. Another example is VeraCode bead and its detection system BeadXpress Reader from Illumina (San Diego, Calif.). The epitope coated beads are incubated with eluate to allow antibodies binding to their corresponding epitopes. Then the antibodies bound to the beads can be quantified by species-specific antibody directly or indirectly conjugated with fluorescent dye for quantification as described elsewhere.

The epitope array contains various epitopes that bind to the antibodies in the eluate. In one embodiment, the epitope used to construct epitope array can be a peptide, a partial protein, complete protein or a fusion protein containing the epitope. The peptide may be chemically synthesized or produced biologically by recombinant protein technology. The partial, complete or fusion proteins can be purified or produced biologically by recombinant protein technology. Both peptide and protein epitopes can be immobilized by various methods as described in the section above on immobilization of antigens onto the epitope carrier and elsewhere regarding protein immobilization methods used in protein array preparation such as Rusmini et al., Protein Immobilization Strategies for Protein Biochips, Biomacromolecules, 2007, 8(6), 1775-1789.

Multiple types of antibodies in the eluate can also be simultaneously detected by mass spectroscopy by identifying and quantifying unique antibody fragments derived from each specific antibody. In one embodiment, the antibodies may need to be subjected to enzyme digestion to generate signature fragments from each epitope-specific antibody before mass spectroscopy analysis. The enzyme can be trypsin or any other enzyme or a mixture of several enzymes that is (are) capable of generating signature fragments for each epitope-specific antibody in the eluate. Other method such as liquid chromatography, gas chromatography, gel electrophoresis may be applied for separation of different signature fragments before subjecting to mass spectroscopy. Various advanced mass spectroscopy technology for analyzing macromolecules such as Surface Enhanced Laser Desorption/Ionization Time of Flight mass spectroscopy (SELDI-ToF) and Matrix-Assisted Laser Desorption/ionization (MALDI) mass spectroscopy can be applied here for detecting and quantifying epitope-specific antibody fragments. Examples of using mass spectroscopy to detect antibody can be found in the following publications: Lewis D A, et al., Characterization of humanized anti-TAC, an antibody directed against the interleukin 2 receptor, using electrospray ionization mass spectrometry by direct infusion, LC/MS, and MS/MS. Anal Chem. 1994 Mar. 1; 66(5):585-95; Matamoros Fernandez L E, et al., Characterization of a recombinant monoclonal antibody by mass spectrometry combined with liquid chromatography. J Chromatogr B Biomed Sci Appl. 2001 Mar. 10; 752(2):247-61; Dick L W, et al., Peptide mapping of therapeutic monoclonal antibodies: improvements for increased speed and fewer artifacts. J Chromatogr B Analyt Techol Biomed Life Sci 2009 Jan. 15; 877(3):230-6; D. Stacy et al., MALDI-TOF mass spectrometry for high-throughput screening of ScFv antibodies targeted to radiation-inducible neoantigens. International Journal of Radiation Oncology Biology Physics, Volume 57, Issue 2, Pages S259-S260; John Vailiere-Douglass et al., Separation and Characterization of an IgG2 Antibody Containing a Cyclic Imide in CDR1 of Light Chain by Hydrophobic Interaction Chromatography and Mass Spectrometry. Anal. Chem., 2008, 80 (9), pp 3168-3174.

In one embodiment, the present invention provides an antibody-based multiplex detection method for identifying one or more epitopes on epitope carriers in one or more samples, the method comprising the steps of: a) contacting each of the samples comprising the epitopes with one or more antibodies specific to the epitopes to be detected; b) washing unbound antibodies away and eluting bound antibodies from the epitope carriers to provide separate antibody eluates; and c) detecting and quantifying antibodies in the antibody eluates to generate separate profiles of antibodies for each of the samples, thereby identifying one or more epitopes in the samples. In general, monoclonal or polyclonal antibodies can be used in this method. In one embodiment, the epitope carriers can be exterior or interior surface of a cytoplasmic membrane, exterior or interior surface of a cell organelle membrane, exterior or interior surface of a cell membrane, a tissue, a tissue section, cells, or an artificial biological surface. The cells can be live cells, apoptotic cells, dead cells or fixed cells. Examples of an artificial biological surface include, but are not limited to, a surface of a culture well, a culture plate, a slide, a bead or a matrix.

In one embodiment, the epitope or epitopes to be detected are present on cell surface antigens, extracellular antigens or intracellular antigens. Alternatively, the epitope or epitopes are present on antigens immobilized onto an artificial biological surface. In another embodiment, the epitope or epitopes are present on different antigen molecules or the same antigen molecule. In general, the epitope or epitopes can be derived from peptide, protein, nucleic acid, carbohydrate or lipid. In the case of peptide epitopes, the epitopes may comprise post-translation modification.

Antibodies in the antibody eluates obtained above may be quantified by detecting epitope-specific antibodies. In one embodiment, such epitope-specific detection comprises detecting epitope-specific antibodies by epitope array. In another embodiment, quantifying antibodies in the antibody eluates comprises detecting species-specific antibodies or uses of mass spectroscopy.

In another embodiment, the present invention provides a multiplex protein detection kit comprising (a) an antibody cocktail; (b) an elution solution; (c) an antibody detection reagent; and (d) an instruction on experimental procedure according to the method described herein. In one embodiment, the antibody detection reagent is an epitope array. In another embodiment, the antibody detection reagent is an enzyme or enzyme mixture to generate unique peptide fragment for each epitope-specific antibody for mass spectroscopy analysis.

### Example 1

### Detection of EGFR-Specific Antibody Eluted from EGFR on Cell Surface

To prove that antibody can be eluted from its epitope and the eluted antibody can be quantified, a murine monoclonal anti-human EGFR antibody (Leinco, St Louis, Mo.) was used on A431 and Hela human cell lines. Confluent A431 and Hela cells each grown on a 6-well dish were first chilled on ice. After aspirating old medium from each well, 0.5 mL of cold new medium (DMEM with 10% FBS) was added into a well as control, 0.5 mL of cold new medium containing 2 μg/mL anti-human EGFR antibody was added into another well. The 6-well dish was then placed on a rocker at 4° C. and rocked for 2 hours. After that, each well were washed three times with cold PBS and eluted with 0.5 mL elution buffer containing 0.5M NaCl, 38 mM Citrate acid, 24 mM NaPO₃, pH 3 at 4° C. for 10 min. Each eluate was neutralized with 20 μl 1M NaCO₃ pH 11 and was diluted 3 fold with 1% non-fat milk in H₂O before subjecting to quantification of the eluted mouse antibody by ELISA shown below.

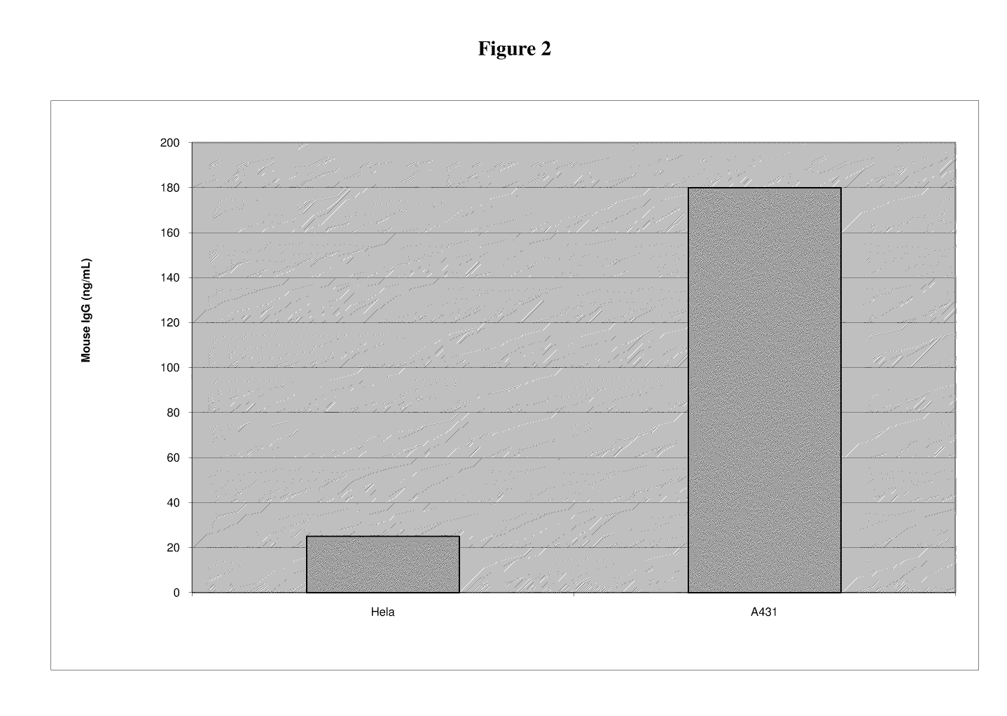

In the ELISA, 0.5 μg/mL Goat Anti-Mouse IgG (Southern Biotech, Birmingham, Ala.) in PBS was used for coating the capture antibody and 1:2000 dilution of HRP conjugated Goat Anti-Mouse kappa antibody (Southern Biotech, Birmingham, Ala.) was used as the detection antibody. A two-fold dilution from 125 ng/mL to 2 ng/mL of the anti-EGFR antibody was used as the standard curve. As shown in FIG. 2, detection of anti-EGFR antibody was achieved in both eluates from A431 and Hela cells incubated with anti-EGFR antibody. In addition, the recovery of anti-EGFR antibody from A431 cells is more than 7 times greater than from Hela cells. Considering the total cell number is 1.9×10⁶ for Hela and 1×10⁶ for A431, the receptor density for A431 cells is 13.7 times greater than Hela cells. This correlates with the publications stating 10-20 fold higher density of EGFR on A431 cells than on Hela cells.

### Example 2

### Simultaneous Detecting EGFR and ErbB2 on Cell Surface

To prove that multiple antigens can be simultaneous detected by their specific antibodies, the murine monoclonal anti-human EGFR antibody and murine monoclonal anti-human ErbB2 antibody (R&D Systems, Minneapolis, Minn.) were used on A431 and Hela human cell lines. Confluent A431 and Hela cells each grown on a 6-well dish were first chilled on ice. After aspirating old medium from each well, 0.5 mL of cold new medium (DMEM with 10% FBS) was added into well #1 as control, 0.5 mL of cold new medium containing 2 μg/mL anti-EGFR antibody was added into well #2 (E1), 0.5 mL of cold new medium containing 2 μg/mL anti-ErbB2 antibody was added into well #3 (E2), 0.5 mL of cold new medium containing 2 μg/mL anti-EGFR antibody and 2 μg/mL anti-ErbB2 antibody was added into well #4 (E1+E2). The 6-well dish was then placed on a rocker at 4° C. and rocked for 2 hours. After that, each well were washed three times with cold PBS and eluted with 0.5 mL elution buffer containing 0.5M NaCl, 38 mM Citrate acid, 24 mM NaPO₃, pH 3 at 4° C. for 10 min. Each eluate was neutralized with 20 μL 1M NaCO₃ pH 11 and was diluted 3 fold with 1% non-fat milk in H₂O before subjecting to quantification of the anti-EGFR antibody and anti-ErbB antibody by ELISA shown below.

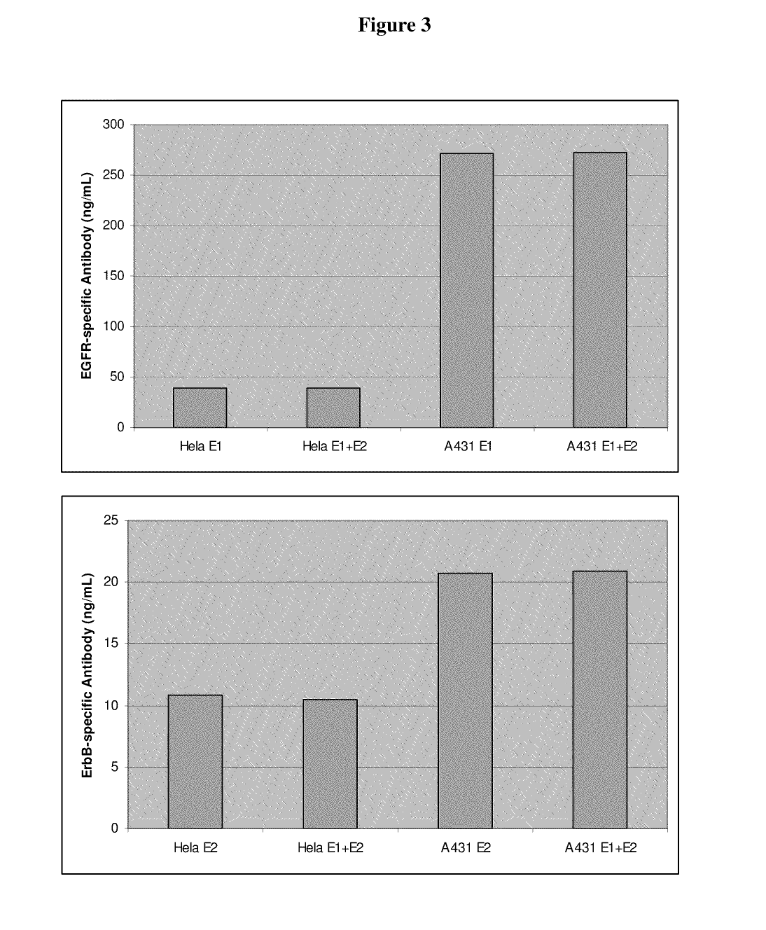

To detect EGFR-specific and ErbB2-specific antibodies by ELISA, a 96-well plate with 1 μg/mL Goat Anti-human Fc antibody (Sigma, St. Louis, Mo.) was first coated, followed by coating with either the chimera protein of EGFR extracellular domain and human Fc (0.25 μg/mL) (R&D Systems, Minneapolis, Minn.) or the chimera protein of ErbB2 extracellular domain and human Fc (0.1 μg/mL) (R&D Systems, Minneapolis, Minn.) to insure full exposure of extracellular domains of EGFR and ErbB2. One hundred μL of diluted eluate was added into the wells coated with either EGFR or ErbB2 for 2 hours incubation. After washing 3 times with PBST (PBS with 0.05% Tween), 1:2000 dilution of HRP conjugated Goat Anti-Mouse kappa antibody (Southern Biotech, Birmingham, Ala.) in PBST with 1% non-fat milk was used as for detection. A two-fold dilution from 125 ng/mL to 2 ng/mL of anti-EGFR antibody or anti-ErbB2 antibody was used as the standard curve. As shown in FIG. 3, detection of almost equal amount of anti-EGFR antibody and anti-ErbB2 antibody in the eluates from the well incubated with anti-EGFR antibody (E1) and the well incubated with both anti-EGFR and anti-ErbB2 antibodies from both A431 and Hela cells (E1+E2) was achieved. It indicates that there is no interference between these two antibodies and they can be used to simultaneously detect EGFR and ErbB2 on a cell surface. In addition, the recovery of anti-EGFR antibody from A431 cells about 7 times greater than from Hela cells was confirmed, similar to example 1 by detecting mouse IgG. The recovery of anti-ErbB2 antibody from A431 cells is 2 fold higher than from Hela cells. After adjusting cell density, A431 has 13.3 fold higher EGFR and 3.8 fold higher ErbB2 on cell surface than Hela cells.

### Example 3

### Cell Surface Receptor Profiling

This example describes the experimental procedure for developing the epitope array and antibody cocktail and using them for multiplex detection of 12 human growth factor receptors that may be present on cell surfaces. The receptors are ErbB-1/EGFR, ErbB-2, ErbB-3, ErbB-4, PDGFRα, PDGFRβ, VEGFR1/Flt-1, VEGFR2/KDR/Flk-1, VEGFR3/Flt-4, Tie-1, Tie-2/Tek, HGF/c-Met.

First, chimera proteins of extracellular domain of each receptor and human Fc portion of IgG (ECD-Fc) will be obtained and coupled to one type of Luminex microsphere of a specific color to generate epitope array for these 12 growth factor receptors. Second, each type of ECD-Fc coupled microsphere will be used to screen for the antibodies that are suitable for this method. Third, an antibody cocktail will be used to develop a protocol for detecting 12 growth factor receptors on cell surface. Finally, the receptor density detected by this method and by conventional flow cytometry will be compared to evaluate the accuracy of this method.

### Development of ECD Epitope Array for 12 Important Growth Factor Receptors

Each of the 12 ECD-Fc chimera proteins will be covalently conjugated into one type of Luminex microsphere of a specific color (Luminex, Austin, Tex.) by following “Sample protocol for two-step carbodiimide coupling of protein to carboxylated microspheres” as recommended by Luminex. For optimization, 1, 5, 25 and 125 μg of each ECD-Fc protein will be used for coupling. To confirm coupling, the ECD-Fc coupled microspheres will then be incubated with a monoclonal antibody to that particular ECD followed by biotinylated anti-mouse IgG Fc antibody (Millipore, Billerica, Mass.) and streptavidin-PE (R&D Systems, Minneapolis, Minn.) before detection by Luminex system. To identify the best coupling condition, two-fold serial dilution of anti-ECD monoclonal antibody from 4 to 0.0625 μg/mL in PBS, 1% BSA solution will be used followed by addition of biotinylated anti-mouse IgG antibody and streptavidin-PE as recommended by Luminex. The microspheres that give the best sensitivity among 4 different levels of ECD-Fc protein will be chosen as its coupling condition. The detailed step-wise procedure is the same as Luminex's sample protocol except without the sample addition step. We will prepare 12 types of microspheres with each type of a specific color carrying the ECD-Fc chimera protein of one of twelve growth factor receptors listed above.

### Generation of an Antibody Cocktail

Each type of ECD-Fc microsphere with a distinct color will be used to screen for the antibody that is suitable for the method. Any antibody qualified for this application needs to have following characteristics: a) specific to ECD of the target receptor; b) can be eluted from the ECD after binding; c) the eluted antibody can be re-natured and quantified by ECD-Fc microspheres on Luminex system; and d) no cross reactivity to other receptors.

To quickly identify antibodies with the first characteristic, commercially available antibodies that can be used in flow cytometry applications will be screened. Flow cytometry applications require antibodies that bind to epitopes present on the extracellular domain of receptors. These antibodies can be obtained from various vendors such as R&D Systems (Minneapolis, Minn.), Abcam (Cambridge, Mass.), LifeSpan BioSciences (Seattle, Wash.), etc. Independently derived monoclonal antibodies against each receptor will be used for the initial screening. Considering the detection limit for Luminex system is usually between 1-10,000 pg/mL, 1, 10, 100, 1000, 10,000 pg/mL of each antibody will be used first to determine if the antibody can successfully recognize ECD of its corresponding receptor on microspheres. To eliminate non-specific binding of detected antibodies, ECD microspheres will be pre-blocked with 1% non-fact milk. The general procedure is 1) incubate each antibody with its ECD microspheres; 2) after washing, add biotinylated anti-species antibody (against the ECD-specific antibody such as anti-mouse or anti-rabbit antibody) (R&D Systems); 3) after washing, add strepavidin-PE conjugate (R&D Systems). The sensitivity and dynamic range of the successful antibody is then determined by titrating antibody by two-fold serial dilution. Considering the receptor density is mostly between 100-100,000 molecules/cell, a million cells will produce 25 pg to 25 ng antibody or 500 pg/mL to 500 ng/mL antibody solution if dissolved in 50 μL volume (standard Luminex assay format) assuming receptor and antibody molecule are in 1:1 ratio. Therefore, the targeted antibody detection dynamic range was set between 500 pg/mL to 500 ng/mL. The antibodies passed this criterion will be moved on for the next elution screening.

Screening Antibodies Elutable from ECD of Receptor

To determine if these antibodies can be eluted from its ECD after binding, a concentration in the high end of the dynamic range identified above was chosen for the test. For testing each antibody, duplicated antibody samples will be used to first incubating with its corresponding ECD-Fc microspheres. After incubation and washing, sample A will be exposed to standard LEAP elution buffer (low pH and high salt) for 10 minutes to elute antibody molecules from microspheres while sample B will remain in PBS, 1% BSA solution. The antibody-containing elution buffer will then be removed from sample A to derive antibody stripped microspheres. The antibody-stripped microspheres will be neutralized by PBS, 1% BSA solution. The microspheres from both samples will then be quantified for the amount of antibody present on microspheres by biotinylated anti-species antibody and strepavidin-PE conjugate. The antibody is considered elutable from ECD if the stripped microspheres produce <10% signal in comparison to un-stripped microspheres. If >50% antibodies can't be successfully eluted, more stringent elution conditions will be tried before screening for additional antibodies. The antibodies passed this eluting screening will be moved forward to the next screen—capability to renature.

Screening Antibodies Renaturable after Elution

To determine if the antibody after elution can be renatured and subsequently quantified by ECD microspheres, duplicate samples (A & B) of an elutable antibody are prepared in 50× concentration of a detectable concentration by its ECD microspheres shown above. Forty-nine fold of volume of either LEAP elution buffer or PBS will be added into sample A or sample B. After 10 min incubation, the sample with elution buffer is neutralized, adjusted to physiological conditions and concentrated into the same volume as sample B. Both samples will then be subjected to antibody quantification by ECD microspheres. If the sample going through denaturing by elution buffer can produce >50% signal in comparison of its control, the antibody is considered “pass” for this screen. The antibodies passing this screen will be assessed for cross-reactivity.

Screening Antibodies with No Cross-Reactivity

The cross-reactivity screening will be conducted by comparing signals generated from each antibody when incubating with a single type of ECD microspheres (format A) and pooled 12 types of ECD microspheres (format B). The antibody will be considered to have “no cross-reactivity” if it generates similar signal from its corresponding ECD microspheres on both format A and B, and no increased signal from other ECD microspheres in comparison to no antibody control. Otherwise, another antibody will be screened against the same receptor that passed last three screens for the cross-reactivity until finding the one without cross-reactivity.

At the end, one antibody for each receptor is selected based on its passing of four criterions above. 12 of these antibodies will be pooled to make antibody cocktail for experiments described below.

### Application of ECD Epitope Array and Antibody Cocktail to Profile 12 Growth Factor Receptors on Cell Surface

Cells lines that have been reported to express many of these growth factor receptors on either the protein or mRNA level will be used for receptor profiling using our Receptor-targeted Protein Array method. The selected cell lines are as follows: MDA-MB-231, A431, Hela, MRC5, MCF-7, HUVEC, and T47D. Specifically, MDA-MB-231 cells can be used for studying EGFR, VEGFR1, and HGFR; A431 for EGFR, VEGFR1, Tie-1, Tie-2 and HGFR; Hela for EGFR and ErbB-2; MRC-5 for PDGFRα and PDGFRβ; MCF-7 for ErbB-3, ErbB-4, Tie-1 and HGFR; HUVEC for VEGFR1, VEGFR2; T47D for ErbB-3, ErbB-4 and VEGFR3.

To avoid non-receptor specific binding such as Fc receptor mediated binding of antibody to cell surface, the tested cells will be pre-blocked by pre-immune IgG of the same species as IgG in antibody cocktail to block non-specific binding sites on the cell surface before contacting receptor-specific antibodies. The general steps for detecting each of these 12 receptors on cell surface are as follows: 1) pre-block assay cells (1 to 10 million) with pre-immune IgG; 2) incubate cells with antibody cocktail. Our starting concentration of each antibody in the cocktail is set at 1 μg/mL; 3) wash away unbound antibodies; 4) elute antibodies from cells; 5) neutralize eluted samples, adjust salt concentration and concentrate into 50 μL; 6) quantify the amount of eluted antibodies by pooled ECD microspheres using biotionylated anti-species antibodies and strepavidin-PE conjugates on Luminex system.

For each receptor, the relative receptor density will also be quantified by conventional flow cytometry method [36] as well using 2-3 kinds of tested cells expressing the receptor. The general procedure for adherent cells is to lift cells by non-enzyme based cell dissociation solution (Sigma, St Louis, Mo.), incubate cells with receptor-specific antibody and detected by biotinylated anti-species antibody against the receptor-specific antibody followed by addition of fluorescence dye conjugated strepavidin such as strepavidin-FITC and strepavidin-PE (BD, Franklin Lake, N.J.). To avoid variation due to cell passage and culture condition, detection of relative receptor density by both methods will be conducted at the same time using the same cells. The relative receptor level derived from the Receptor-targeted Protein Array method will be compared with the relative receptor level detected by conventional flow cytometry method for us to evaluate the accuracy of the method of our invention.

### REFERENCES

1. Harry Rubin, Antibody elution from red blood cells, J. Clin. Path. 1963, 16, 70-73.
2. C. J. van Oss et al. Elution of blood group antibodies from red cells, Vox Sang. 1981, 40, 367-371.
3. Buffington et al., Antibody elution reagent kit, U.S. Pat. No. 4,426,357.
4. Harlow E, Lane D, Using Antibodies: A Laboratory Manual. Cold Spring Harbor, N.Y., Cold Spring Harbor Laboratory Press.
5. Pirici D., et al. Antibody elution method for multiple immunohistochemistry on primary antibodies raised in the same species and of the same subtype, Journal of Histochemistry & Cytochemistry, 2009, 57(6): 567-575.

What is claimed is:

1. An antibody-based multiplex detection method for identifying one or more epitopes on epitope carriers in one or more samples, the method comprising the steps of:
a) contacting each of the samples comprising the epitopes with one or more antibodies specific to the epitopes to be detected;
b) washing unbound antibodies away and eluting bound antibodies from the epitope carriers to provide separate antibody eluates; and
c) detecting and quantifying antibodies in the antibody eluates to generate separate profiles of antibodies for each of the samples, thereby identifying one or more epitopes in the samples.

2. The method of claim 1, wherein the epitope carriers are selected from the group consisting of exterior or interior surface of a cytoplasmic membrane, exterior or interior surface of a cell organelle membrane, a tissue, a tissue section, cells, and an artificial biological surface.

3. The method of claim 2, wherein the artificial biological surface is a surface of a culture well, a culture plate, a slide, a bead or a matrix.

4. The method of claim 2, wherein the cells are live cells, apoptotic cells, dead cells or fixed cells.

5. The method of claim 1, wherein the epitope or epitopes are present on antigens selected from the group consisting of cell surface antigens, intracellular antigens, and extracellular antigens.

6. The method of claim 1, wherein the epitope or epitopes are present on antigens immobilized onto an artificial biological surface.

7. The method of claim 1, wherein the epitope or epitopes are present on different antigen molecules or the same antigen molecule.

8. The method of claim 1, wherein the epitope or epitopes are derived from peptide, protein, nucleic acid, carbohydrate or lipid.

9. The method of claim 8, wherein the peptide epitope or epitopes comprise post-translation modification.

10. The method of claim 1, wherein quantifying antibodies in the antibody eluates comprises detecting epitope-specific antibodies.

11. The method of claim 10, wherein the detection of epitope-specific antibodies comprises using epitope array.

12. The method of claim 1, wherein quantifying antibodies in the antibody eluates comprises detecting species-specific antibodies or uses of mass spectroscopy.

13. The method of claim 1, wherein the antibodies are monoclonal antibodies or polyclonal antibodies.

14. A multiplex protein detection kit comprising (a) an antibody cocktail; (b) an elution solution; (c) an antibody detection reagent; and (d) an instruction on experimental procedure according to the method of claim 1.

15. The detection kit of claim 14, wherein the antibody detection reagent is an epitope array.
